(19) United States
(12) Patent Application Publication
(10) Pub. No.: US 2012/0156314 A1
(43) Pub. Date: Jun. 21, 2012

(54) 11 beta, 13-DIHYDROHELENALIN DERIVATIVES AND USES THEREOF AS BOMBESIN RECEPTOR SUBTYPE 3 AGNOISTS

(75) Inventors: Klim KING, Taipei Hsien (TW); Shoei-Sheng Lee, Taipei Hsien (TW); Ching-Kuo Lee, Taipei Hsien (TW); Hual-Cheng Lee, Taipei Hsien (TW)

(73) Assignee: VIOGENE-BIOTEK CORPORATION, Taipei Hsien (TW)

(21) Appl. No.: 12/968,306

(22) Filed: Dec. 15, 2010

(51) Int. Cl. A61K 36/28; A61P 9/12; A61P 3/08; A61P 3/04; A61K 31/343; A61P 3/00

(52) U.S. Cl. 424/725; 514/468

(57) ABSTRACT

The present invention relates to compounds of formual (I):

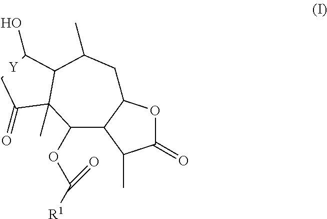

wherein Y is a single bond or double bound; and R¹ is —C(CH₃)C₂H₅ or —C(CH₃)═CHCH₃, when Y is a single bound; R¹ is —C(CH₃)═CH₂, is —C(CH₃)₂, —C(CH₃)═CHCH₃, —C(CH₃)C₂H₅, or —CH₂C(CH₃)₂, when Y is a double bound. These compounds are bombesin receptor subtype 3 (BRS3) modulating agents, more specifically that they are BRS3 agonists. The invention is also related to the use of such compounds to treat diseases associated with inappropriate BRS3 activity.

### FIELD OF THE INVENTION

The present invention is related to extracts from Centipeda minima (L.) A. Braun et Aschers. (Compositae) and compounds identified therefrom, which are capable of activating bombesin receptor subtype 3 (BRS3), i.e. nonpeptide, small molecular BRS3 agonists.

### BACKGROUND OF THE INVENTION

Bombesin receptor subtype 3 (BRS3) is a G-protein coupled receptor with a wide variety of physiological function, including regulation of energy homeostasis and feeding behavior in rodents. Though peptide ligand acting as an agonist at this peptidergic receptor has been identified for more than 10 years, nonpeptide ligands capable of activating BRS3 are still not available so far.

Since central nerve system is one of the major organs where BRS3 is expressed, there is an urgent need for small molecular bombesin ligands that are able to pass through blood brain barrier to allow pharmacological investigation of the function of BRS3 and may further be developed into therapeutic agents. Furthermore, the development of peptides as drugs is problematic as a result of poor oral and tissue absorption, rapid proteolytic cleavage and poor shelf stability.

### SUMMARY OF THE INVENTION

An primary objective of the present invention is to provide metabolically stable and small molecular weight non-peptide compounds which are capable of activating bombesin receptor subtype 3 (BRS3).

Another objective of the present invention is to provide extracts from Centipeda minima (L.) A. Braun et Aschers. (Compositae) which are capable of activating bombesin receptor subtype 3 (BRS3).

Still another objective of the present invention is to provide use of the compound, or a pharmaceutically acceptable salt thereof, and the extract of the present invention in the treatment of a BRS3 mediated disease. An embodiment of such use is a method for treating a BRS3 mediated disease in a patient comprising administering to the patient, as an agonist, an amount of the extract of the present invention, or the compound of the present invention, or a pharmaceutically acceptable salt thereof, effective to modulate a BRS3-mediated biological activity. Another embodiment of such use is a pharmaceutical composition for treating a BRS3 mediated disease in a patient comprising, as an agonist, the extract of the present invention of the compound of the present invention, or a pharmaceutically acceptable salt thereof.

Two compounds having the following structures (A) and (B) are isolated from the extract of Centipeda minima (L.) A. Braun et Aschers. (Compositae):

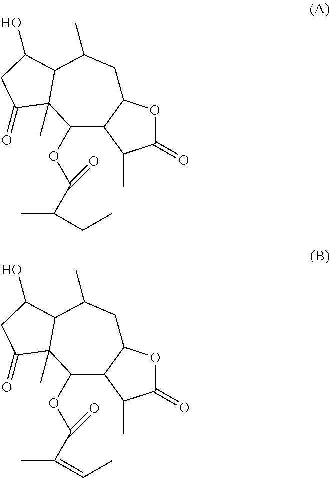

both of which are BRS3 agonists.

The present invention also isolated another five compounds from the extract of Centipeda minima (L.) A. Braun et Aschers. (Compositae) having the following structures (1)-(5):

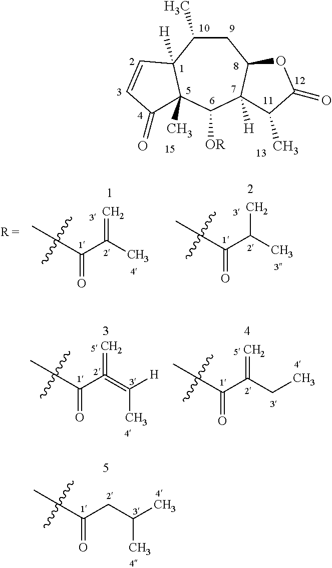

all of which are BRS3 agonists.

The extract of Centipeda minima (L.) A. Braun et Aschers. (Compositae) of the present invention is prepared by a process comprising the following step:

a) Extracting Centipeda minima (L.) A. Braun et Aschers. (Compositae) with a polar solvent, preferably ethanol, ethanol aqueous solution, or ethyl acetate, and more preferably ethanol or 95% ethanol aqueous solution.

Preferably, the process further comprises: b) partitioning the 95% ethanol extract from step a) with ethyl acetate, and recovering ethyl acetate fraction. More preferably, partitioning in step b) comprises drying the 95% ethanol extract; suspending the dried ethanol extracts in methanol or methanol aqueous solution; partitioning the methanol suspension with hexane, discarding the hexane layer; drying the resultant methanol suspension and re-suspending the dried suspension in water; partitioning the water suspension with ethyl acetate; and concentrating the resultant ethyl acetate layer to obtain the ethyl acetate fraction.

Preferably, the process further comprises: c) introducing the ethyl acetate fraction to a normal phase chromatography column; d) eluting with a first eluent of a non-polar solvent such as hexane and with a second eluent in sequence, wherein the second eluent having a polarity of about 30-50 vol % ethyl acetate in hexane; and e) collecting a second eluate from the elution of the column with the second eluent, and removing the second eluent from the second eluate. More preferably, the column is further eluted with a mixed solvent of ethyl acetate and hexane having 5-20 vol % of ethyl acetate after the elution of the column with the first eluent and before the elution of the column with the second eluent.

Preferably, the second eluate from the elution of the column with the second eluent comprises a compound having the structure (A) or (B) defined above, and more preferably, the second eluate comprises both the compounds having the structures (A) and (B)

Preferably, the second eluate from the elution of the column with the second eluent comprises a compound having the structure (1), (2), (3), (4), or (5), and more preferably, the second eluate comprises all the compounds having the structures (1) to (5).

Preferably, the second eluate from the elution of the column with the second eluent comprises all the compounds having the structures (A), (B), (1), (2), (3), (4), and (5).

### DETAILED DESCRIPTION OF THE INVENTION

BRS3 is a peptidergic GPCR that plays a role in physiological regulation of energy metabolism and feeding behavior, however, nonpeptide agonist that are able to activate this receptor is still not available. Animal missing this receptor display broad range of metabolic disorders including increasing food intake, obesity, glucose intolerance and hypertension, implying an important role of BRS3 in energy homeostasis. Since BRS3 is expressed predominantly in hypothalamus and pituitary gland, finding a small molecule agonist that can crossed blood brain barrier is essential for developing therapeutics to treat BRS3 mediated metabolic disorders and obtaining tool to study the underlying physiological mechanism.

### DEFINITIONS

“C1-C6 Alkyl” means a linear saturated monovalent hydrocarbon radical of one to six carbon atoms, or a cyclic or branched saturated monovalent hydrocarbon radical of three to six carbon atoms, unless otherwise stated, e.g., methyl, ethyl, propyl, 2-propyl, butyl, and the like.

“C1-C6 Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“C2-C6 alkenyl” means a linear monovalent hydrocarbon radical of two to six carbon atoms containing an unsaturated double bound or a branched monovalent hydrocarbon radical of three to six carbon atoms containing an unsaturated double bound.

“C2-C6 alkenylene” means a linear divalent hydrocarbon radical of two to six carbon atoms containing an unsaturated double bound or a branched divalent hydrocarbon radical of three to six carbon atoms containing an unsaturated double bound.

“Treating” and “Treatment”, includes any effect, e.g., lessening, reducing, modulating, or eliminating, that results in the improvement of the condition, disease, disorder, etc. and includes preventative and reactive treatment.

“Pharmaceutically-acceptable salt” means a salt prepared by conventional means, and are well known by those skilled in the art. The “pharmaceutically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

It will be noted that the structure of some of the compounds of the invention includes asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Alkenes can include either the E- or Z-geometry, where appropriate.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyl-oxyalkyl (for example, pivaloyloxymethyl). Amines have been masked as arylcarbonyloxymethyl substituted derivatives, which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

“EC₅₀ of an agent” included that concentration of an agent at which a given activity, including binding of sphingosine or other ligand of an S1P receptor and/or a functional activity of a S1P receptor (e.g., a signaling activity), is 50% maximal for that S1P receptor. Stated differently, the EC₅₀ is the concentration of agent that gives 50% activation, when 100% activation is set at the amount of activity of the BRS3 which does not increase with the addition of more ligand/agonist and 0% activation is set at the amount of activity in the assay in the absence of added ligand/agonist.

“Purified” and like terms relate to the isolation of a molecule or compound in a form that is substantially free of contaminants normally associated with the molecule or compound in a native or natural environment.

An “effective amount” includes an amount sufficient to produce a selected effect. For example, an effective amount of a BRS3 agonist is an amount that increases the cell signaling activity of the BRS3.

“Pharmaceutically acceptable” include molecular entities and compositions that do not produce an adverse, allergic or other untoward reaction when administered to an animal, or a human, as appropriate.

“Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The present invention will be better understood through the following examples which are merely illustrative, not for limiting the scope of the present invention.

### Experiments

### Preparation of Extracts, MB1136-E, MB1136-A-01 and MB1136-E-EA

The dried and ground whole plant of Centipeda minima (L.) A. Braun et Aschers. (Compositae) (2 kg) was extracted with 20 L of 95% EtOH at room temperature for 16 hr, followed by filtration and concentration under reduced pressure to obtain dried ethanol extract (330 g), MB1136-E. The dried ethanol extract (330 g) were resuspended into 1.65 L 90% methanol. The suspension was subjected to hexane partitioned by mixing with equal volume of hexane for three times, wherein the hexane layer was discarded. The resultant 95% methanol suspension from the partitioning was concentrated, dried, and re-suspended in water (1.2 L). The water suspension was further partitioned by ethyl acetate by mixing with equal volume of ethyl acetate for three times to generate ethyl acetate layers. The ethyl acetate layers were collected, and dried in vacuo to obtain ethyl acetate extract (50.3 g), MB1136-E-EA.

An ethyl acetate extract (MB1136-A-01) of MB1136 was prepared by repeating the procedures of preparing MB1136-E, except that the 95% ethanol was replaced with ethyl acetate.

Silica gel chromatography of MB1136-E-EA

Ethyl acetate fraction, containing the most of the activity, was further subjected to chromatography on an open gravity column packed with 70-230 mesh silica.gel 60 (E. Merck, Darmstadt, Federal Republic of Germany) in an amount of 3 times by dry weight of the concentrate. The column was washed with hexane (5 bed volumes), then eluted by steps of increasing ethyl acetate in hexane (5, 10, 20, 30, 40, 50, 75 and 100%), followed by eluting with 20% and 50% of methanol in ethyl acetate, wherein the amounts of the eluents were 5 bed volumes. This step of separation resolved the extract into 35 fractions, the amount of compound in each fraction was ranged from 25 g to 200 mg. Following BRS3 agonist activity assay for each fraction, the chromatogram profile revealed two activity peaks, the major one is fraction 16 and the minor one is fraction 19, which were eluted at 30% and 50% ethyl acetate in hexane, respectively. The agonist activity of the ethyl acetate layer, fraction 15, 16, 17, 18, 19 and 20 were subject to dose dependent analysis to measure the affinity (EC₅₀) and maximal activity (Eₘₐₓ) for BRS3 activation.

### Separation of Fraction 16

¹H and ¹³C NMR spectra were recorded on a Bruker AV400 spectrometer (CDCl₃, δ_{H} 7.24 and δ_{c} 77.0 ppm). The UV spectra were measured on a Hitachi U-2001 UV Spectrophotometer and a JASCOJ-720 spectropolarimeter. The HPLC-DAD was composed of an Agilent 1100 liquid chromatographs with LC quaternary pumps, equipped with a Rheodyne model 7725i injection valve linking to a 20 μL injection loop, a Bruker photodiode array detector. MS data were measured on an Esquire 2000 ion trap mass spectrometer (Bruker Daltonik) with electrospray ion source. TLC analysis was performed on silica gel plates (KG60-F₂₅₄, Merck).

n-Hexane, chloroform and methanol (CAS and HPLC grade) were purchased from Mallinckrodt (KY, USA). Ethanol (95%) was supplied by Taiwan Tobacco and Liquor Corporation. CDCl₃ (99.8%) was purchased from Cambridge (MA, USA) and deionized water was prepared from a Barnstead water purification system (Dubuque, Iowa, USA).

Fraction 16 (50.2 mg) in MeOH-dichloromthane (1:1, 1 mL) was passed through a Sephadex LH-20 column (70 mL, MeOH—CH₂Cl₂ 1:1) to give seven fractions. Fraction 5 (32.4 mg) was further fractionated into five subfractions, using the same column and chromatographic conditions as indicated above. Subfraction 3 (11.7 mg) was chromatographed over a semi-preparative RP-18 column (Phenomenex Prodigy 5μ ODS3, 100A, 250×10 mm) under the following conditions, MeCN—H₂O (43:57), flow rate 3.5 mL/min, injection volume 55 μL×2 (c=10.7 mg/110 μL MeOH), and detection at 223 nm, to give five compounds (1-5) in the amount of 0.2, 1.8, 6.2, 6.9, and 1.5 mg, respectively. The retention time of each compound was 22.26 min (1), 25.40 min (2), 34.40 (3), 38.24 min (4), and 40.71 min (5), respectively, under the folloing chromatographic conditions: RP-18 column (Phenomenex Prodigy 5μ ODS3, 100A, 250×4.6 mm), MeCN—H₂O (43:57), injection volume 1 μL (2.3 mg/60 μL MeOH), flow rate 0.7 mL/min, and detection at 223 nm.

### Structure Analysis of Compounds (1-5)

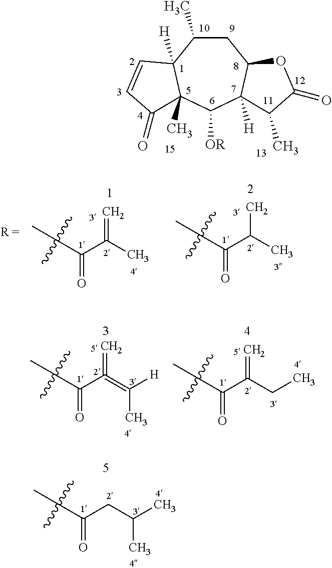

ESIMS data of compounds (1-5)

### Compound (1): Arnicolide D, C₁₉H₂₄O₅

### Positive ESI/MS/MS: m/z 355 [M+Na]⁺, 269.

### Compound (2): Arnicolide C, C₁₉H₂₆O₅

### Positive ESI/MS/MS: m/z 357 [M+Na]⁺, 269.

### Compound (3): Microhelenin C, C₂₀H₂₆O₅

### Positive ESI/MS/MS: m/z 369 [M+Na]⁺, 269.

### Compound (4): Microhelenin B, C₂₀H₂₈O₅

### Positive ESI/MS/MS: m/z 371 [M+Na]⁺, 269.

### Compound (5): Arnicolide B, C₂₀H₂₈O₅

### Positive ESI/MS/MS: m/z 371 [M+Na]⁺, 269.

The ¹H NMR spectra of compounds (1-5) showed common signals for the α and β protons in an enone system, δ 7.64 (dd, J=1.7, 6.0 Hz, H-2) and 6.03 (dd, J=3.0, 6.0 Hz, H-3), a lactonic proton around δ 4.72 (1H, ddd, J=1.8, 5.9, 6.5 Hz, H-8), an ester proton around δ 5.40 (1H, br s, H-6), one methyl singlet around δ 1.01 (3H, s, H-15), and two methyl doublets around δ1.50 (3H, d, J=7.4 Hz, H-13) and 1.21 (3H, d, J=6.7 Hz, H-14) (Table 1). These data will constitute a basic skeleton of 6-hydroxy-4-oxo-2-pseudoguaien-12,8-olide [1]. This presence of such skeleton in (4) was confirmed by a homo COSY spectrum, which showed the following correlations: δ 6.03 dd (H-3)→7.64 dd (H-2)→3.03 ddd (H-1)→2.19 m (H-10)→2.45 ddd and 1.62 ddd (H-9)→4.72 ddd (H-8) br dd (H-7)→5.40 br s (H-6), and δ 2.79 (H-7)→3.06 dq (H-11)→1.50 d (H-13), and δ 2.19 (H-10)→1.21 d (H-14). Besides the skeleton protons, nine protons' signals were observed and their coupling relationships were clarified by the COSY spectrum as follows: δ 1.02 d (3H) (H-5′)→2.22 m (1H) (H-2′)→1.52 m/1.34 ddq (each 1H) (H-3′)→0.80 t (3H) (H-4′). The latter correlation will constitute a 2-methyl-butanoyl moiety. Accordingly, compound 4 is likely to be microhelenin B [1]. This suggestion was confirmed by the ESIMS spectrum which displayed the [M+Na]+ at m/z 371 and fit the molecular formula C₂₀H₂₈O₅. Other supportive evidences included the UV (λmax 223 nm), ¹³C NMR (Table 1), NOESY, HMQC and HMBC shown in the following formula. The ester linkage at C-6 was confirmed by the observation of the correlation between H-6 (δ 5.40) and C-1′ (δ 175.2).

TABLE I

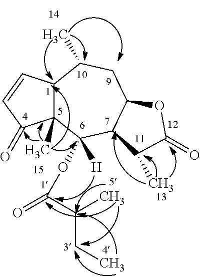

Key HMBC correlations of microhelenin B (4)
¹H- and ¹³C- NMR data (δ/ppm) of 1-4, and HMBC correlations of microhelenin
B (4) (CDCl3 , 400 MHz)

| | 4 | | HMBC of 4 (J = 8 Hz) | |
|---|---|---|---|---|
| Position | δ_{H} (multi.) (J in Hz)^{a} | δ_{C} (multi.)^{b} | δ_{H} | Correlations (C#) |
| 1 | 3.03 ddd (1.7, 3.0, 10.6) | 53.99 d | 3.03 | C-2, C-3, C-5, C-9, C-10 |
| 2 | 7.64 dd (1.7, 6.0) | 161.93 d | 7.64 | C-1, C-3, C-4, C-5 |
| 3 | 6.03 dd (3.0, 6.0) | 129.42 d | 6.03 | C-1, C-2, C-4 |
| 4 | | 209.30 s | | |
| 5 | | 54.76 s | | |
| 6 | 5.40 br s | 71.64 d | 5.40, | C-1, C-1′, C-4, C-5, C-7, C-8, |
| | | | | C-11, C-15 |
| 7 | 2.79 dd (6.5, 10.2) | 48.99 d | 2.79 | C-5, C-6, C-11, C-12 |
| 8 | 4.72 ddd (1.8, 5.9, 6.5) d | 79.42 | 4.72 | C-6, C-9, C-10, C-11 |
| 9 | 2.45 ddd (2.2, 5.9, 15.3) | 40.99 t | 2.45 | C-1, C-7, C-8, C-10, C-14 |
| | 1.62 ddd (1.8, 11.1, 15.3) | | 1.62 | C-1, C-10 |
| 10 | 2.19 m | 25.79 d | 2.19 | C-6, C-7, C-12, C-13 |
| 11 | 3.06 dq (7.4, 10.2) | 40.49 d | 3.06 | |
| 12 | | 178.89 s | | |
| 13 | 1.50 d (7.4) | 10.86 q | 1.50 | C-7, C-11, C-12 |
| 14 | 1.21 d (6.7) | 19.68 q | 1.21 | C-1, C-9, C-10 |
| 15 | 1.01 s | 17.55 q | 1.01 | C-4, C-5, C-6 |
| 1′ | | 175.19 s | | |
| 2′ | 2.22 m | 40.90 d | 2.22 | C-1′, C-3′, C-4′, C-5′ |
| 3′ | 1.52 m | 26.54 t | 1.52 | C-1′, C-2′, C-4′, C-5′ |
| | 1.34 ddq (7.1, 7.4, 14.2) | | 1.34 | C-1′, C-2′, C-4′, C-5′ |
| 4′ | 0.80 t (7.4) | 11.30 q | 0.80 | C-2′, C-3′ |
| 5′ | 1.02 d (6.8) | 16.18 q | 1.02 | C-1′, C-2′, C-3′ |

^{a}Other ¹H NMR data of 1-3 and 5: 1: δ 5.46 (1H, br s, H-6), 2.91 (1H, dd, J = 6.5, 10.2 Hz, H-7), 1.53 (3H, d, J = 7.4 Hz, H-13), 5.86 (1H, q, J = 1.0 Hz, H-3′), 5.49 (1H, dq, J = 1.1, 1.5 Hz, H-3′) and 1.82 (3H, br s, H-4′); 2: δ 2.39 (1H, m, H-2′), 1.06 (3H, d, J = 6.8 Hz, H-3′) and 1.02 (3H, d, J = 7.0 Hz, H-3″); 3: δ 5.48 (1H, br s, H-6), 2.91 (1H, dd, J = 6.5, 10.2 Hz, H-7), 1.54 (3H, d, J = 7.4 Hz, H-13), 6.01 (1H, qq, J = 1.3, 7.3 Hz, H-3′), 1.88 (3H, dq, J = 1.3, 7.3 Hz, H-4′) and 1.71 (3H, q-like, J = 1.3 Hz, H-5′); 5: δ 2.07 (1H, d, J = 7.5 Hz, H-2′), 2.05 (1H, d, J = 7.5 Hz, H-2), 1.95 (1H, m, H-3′), 0.88 (6H, d, J = 6.6 Hz, H-4′and H-4″).
^{b}Other ¹³C NMR data of 2-3 and 5: 2: δ 175.6 (s, C-1′), 33.9 (d, C-2′), 18.9 (q, C-3′), 18.6 (q, C-3″); 3: δ 166.3 (s, C-1′), 127.3 (s, C-2′), 139.1 (d, C-3′), 15.7 (q, C-4′), 20.5 (q, C-5′); 5: δ 171.7 (s, C-1′), 43.4 (t, C-2′), 25.7 (d, C-3′), 22.4 (q, C-4′), 22.3 (q, C-4″).

The ESIMS of compound (1) showed the [M+Na]⁺ at m/z 355, which gave a molecular formula C₁₉H₂₄O₅ incorporating with the ¹H NMR spectral data (Table 1). Besides the signals for the basic skeleton as those in (4), the ¹H NMR spectrum of (1) exhibited signals for a 2-methylpropenoyl moiety, δ 5.86 (1H, q, J=1.0 Hz, H-3′), 5.49 (1H, dq, J=1.1, 1.5 Hz, H-3′) and 1.82 (3H, br s, H-4′). Accordingly, compound (1) was established as arnicolide D [2]. The ESIMS of compound (2) showed the [M+Na]⁺ at m/z 357, which gave a molecular formula C₁₉H₂₆O₅ incorporating with the ¹H and ¹³C NMR spectral data (Table 1). Besides the signals for the basic skeleton as those in (4), the ¹H NMR spectrum of (2) exhibited signals for a 2-methylpropanoyl moiety, δ 2.39 (1H, m, H-2′), 1.06 (3H, d, J=6.8 Hz, H-3′) and 1.02 (3H, d, J=7.0 Hz, H-4′). Accordingly, compound (2) was established as arnicolide C [2]. The ESIMS of compound (3) showed the [M+Na]⁺ at m/z 369, which gave a molecular formula C₂₀H₂₆O₅ incorporating with the ¹H and ¹³C NMR spectral data (Table 1). Besides the signals for the basic skeleton as those in (4), the ¹H NMR spectrum of (3) exhibited signals for a 2-methyl-2-butenoyl (tigloyl) moiety, δ 6.01 (1H, qq, J=1.3, 7.3 Hz, H-3′), 1.88 (3H, dq, J=1.3, 7.3 Hz, H-4′) and 1.71 (3H, q-like, J=1.3 Hz, H-5′). Accordingly, compound (3) was established as microhelenin C [1]. The ESIMS of compound (5) showed the [M+Na]⁺ at m/z 371, which gave a molecular formula C₂₀H₂₈O₅ incorporating with the ¹H and ¹³C NMR spectral data (Table 1). Besides the signals for the basic skeleton as those in (4), the ¹H NMR spectrum of (3) exhibited signals for a 3-methylbutanoyl moiety, δ 2.07 (1H, d, J=7.5 Hz, H-2′), 2.05 (1H, d, J=7.5 Hz, H-2′), 1.95 (1H, m, H-3′), 0.88 (6H, t, J=6.6 Hz, H-4′ and H-5′). Accordingly, compound (5) was established as arnicolide B [2].

### Separation of Fraction 19

2.03 g of fraction 19 was fractionated into 113 fractions plus pre-fraction (non retained polar compounds) and post-fraction (rinsing of column after fractionation) according to the method listed in the following Table S1. Comprehensive tables of fractionations with amount of fraction, amount of aliquot, plate position, and fraction number are listed in Table S2.

TABLE S1

| HPLC System | SEPBOX (Fa. SEPIAtec) | | |
|---|---|---|---|
| Stationary Phase | Kromasil C18 250 × 50 mm, 10 μm | | |
| Injected Amount | 2.03 g | | |
| Flow rate | 109 ml/min | | |
| Detection | ELSD (Sedex 75), UV (Merck, 250 nm) | | |
| Solvents | A: Water | | |
| | B: Methanol | | |
| Gradient | Time [min] | % A | % B |
| | 0.00 | 45 | 55 |
| | 45.0 | 0 | 100 |
| | 50.0 | 0 | 100 |

TABLE S2

| | Fraction | Aliquot | | |
|---|---|---|---|---|
| Comment | [mg] | [mg] | Plate | Position |
| Prefraction | 126.2 | 0.5 | C-0352-N-1 | A01 |
| Fraction_1 | 10.9 | 0.5 | C-0352-N-1 | B01 |
| Fraction_2 | 18.2 | 0.5 | C-0352-N-1 | C0 |
| Fraction_3 | 24.1 | 0.5 | C-0352-N-1 | D01 |
| Fraction_4 | 25.4 | 0.5 | C-0352-N-1 | E01 |
| Fraction_5 | 24.3 | 0.5 | C-0352-N-1 | F01 |
| Fraction_6 | 18.1 | 0.5 | C-0352-N-1 | G01 |
| Fraction_7 | 9.3 | 0.5 | C-0352-N-1 | H01 |
| Fraction_8 | 6.4 | 0.5 | C-0352-N-1 | A02 |
| Fraction_9 | 4.8 | 0.5 | C-0352-N-1 | B02 |
| Fraction_10 | 5.3 | 0.5 | C-0352-N-1 | C02 |
| Fraction_11 | 6.1 | 0.5 | C-0352-N-1 | D02 |
| Fraction_12 | 6.7 | 0.5 | C-0352-N-1 | E02 |
| Fraction_13 | 6 | 0.5 | C-0352-N-1 | F02 |
| Fraction_14 | 5.6 | 0.5 | C-0352-N-1 | G02 |
| Fraction_15 | 4.4 | 0.5 | C-0352-N-1 | H02 |
| Fraction_16 | 3.2 | 0.5 | C-0352-N-1 | A03 |
| Fraction_17 | 4.5 | 0.5 | C-0352-N-1 | B03 |
| Fraction_18 | 4.2 | 0.5 | C-0352-N-1 | C03 |
| Fraction_19 | 3.5 | 0.5 | C-0352-N-1 | D03 |
| Fraction_20 | 2.3 | 0.5 | C-0352-N-1 | E03 |
| Fraction_21 | 2.7 | 0.5 | C-0352-N-1 | F03 |
| Fraction_22 | 3.6 | 0.5 | C-0352-N-1 | G03 |
| Fraction_23 | 3.7 | 0.5 | C-0352-N-1 | H03 |
| Fraction_24 | 6.6 | 0.5 | C-0352-N-1 | A04 |
| Fraction_25 | 6.1 | 0.5 | C-0352-N-1 | B04 |
| Fraction_26 | 5.4 | 0.5 | C-0352-N-1 | C04 |
| Fraction_27 | 5.3 | 0.5 | C-0352-N-1 | D04 |
| Fraction_28 | 6.8 | 0.5 | C-0352-N-1 | E04 |
| Fraction_29 | 5.9 | 0.5 | C-0352-N-1 | F04 |
| Fraction_30 | 7.1 | 0.5 | C-0352-N-1 | G04 |
| Fraction_31 | 9 | 0.5 | C-0352-N-1 | H04 |
| Fraction_32 | 6.4 | 0.5 | C-0352-N-1 | A05 |
| Fraction_33 | 3.4 | 0.5 | C-0352-N-1 | B05 |
| Fraction_34 | 3 | 0.5 | C-0352-N-1 | C05 |
| Fraction_35 | 4 | 0.5 | C-0352-N-1 | D05 |
| Fraction_36 | 4.3 | 0.5 | C-0352-N-1 | E05 |
| Fraction_37 | 5.5 | 0.5 | C-0352-N-1 | F05 |
| Fraction_38 | 6.5 | 0.5 | C-0352-N-1 | G05 |
| Fraction_39 | 6.1 | 0.5 | C-0352-N- l | H05 |
| Fraction_40 | 6.7 | 0.5 | C-0352-N-1 | A06 |
| Fraction_41 | 9.5 | 0.5 | C-0352-N-1 | B06 |
| Fraction_42 | 6.9 | 0.5 | C-0352-N-1 | C06 |
| Fraction_43 | 6.4 | 0.5 | C-0352-N-1 | D06 |
| Fraction_44 | 11.2 | 0.5 | C-0352-N-1 | E06 |
| Fraction_45 | 11.1 | 0.5 | C-0352-N-1 | F06 |
| Fraction_46 | 5.5 | 0.5 | C-0352-N-1 | G06 |
| Fraction_47 | 6.7 | 0.5 | C-0352-N-1 | H06 |
| Fraction_48 | 5.6 | 0.5 | C-0352-N-1 | A07 |
| Fraction_49 | 4.4 | 0.5 | C-0352-N-1 | B07 |
| Fraction_50 | 4 | 0.5 | C-0352-N-1 | C07 |
| Fraction_51 | 4.8 | 0.5 | C-0352-N-1 | D07 |
| Fraction_52 | 9 | 0.5 | C-0352-N-1 | E07 |
| Fraction_53 | 5.8 | 0.5 | C-0352-N-1 | F07 |
| Fraction_54 | 3.4 | 0.5 | C-0352-N-1 | G07 |
| Fraction_55 | 3.7 | 0.5 | C-0352-N-1 | H07 |
| Fraction_56 | 3.3 | 0.5 | C-0352-N-1 | A08 |
| Fraction_57 | 5.1 | 0.5 | C-0352-N-1 | B08 |
| Fraction_58 | 7 | 0.5 | C-0352-N-1 | C08 |
| Fraction_59 | 3.8 | 0.5 | C-0352-N-1 | D08 |
| Fraction_60 | 3.1 | 0.5 | C-0352-N-1 | E08 |
| Fraction_61 | 2.9 | 0.5 | C-0352-N-1 | F08 |
| Fraction_62 | 3.9 | 0.5 | C-0352-N-1 | G08 |
| Fraction_63 | 21.9 | 0.5 | C-0352-N-1 | H08 |
| Fraction_64 | 16.2 | 0.5 | C-0352-N-1 | A09 |
| Fraction_65 | 24.3 | 0.5 | C-0352-N-1 | B09 |
| Fraction_66 | 15.3 | 0.5 | C-0352-N-1 | C09 |
| Fraction_67 | 6 | 0.5 | C-0352-N-1 | D09 |
| Fraction_68 | 4.1 | 0.5 | C-0352-N-1 | E09 |
| Fraction_69 | 2.7 | 0.5 | C-0352-N-1 | F09 |
| Fraction_70 | 3.7 | 0.5 | C-0352-N-1 | G09 |
| Fraction_71 | 4.5 | 0.5 | C-0352-N-1 | H09 |
| Fraction_72 | 4.6 | 0.5 | C-0352-N-1 | A10 |
| Fraction_73 | 4.1 | 0.5 | C-0352-N-1 | B10 |
| Fraction_74 | 4.9 | 0.5 | C-0352-N-1 | C10 |
| Fraction_75 | 3.9 | 0.5 | C-0352-N-1 | D10 |
| Fraction_76 | 2.9 | 0.5 | C-0352-N-1 | E10 |
| Fraction_77 | 2.9 | 0.5 | C-0352-N-1 | F10 |
| Fraction_78 | 3.5 | 0.5 | C-0352-N-1 | G10 |
| Fraction_79 | 2.9 | 0.5 | C-0352-N-1 | H10 |
| Fraction_80 | 3.5 | 0.5 | C-0352-N-2 | A01 |
| Fraction_81 | 3.3 | 0.5 | C-0352-N-2 | B01 |
| Fraction_82 | 3.5 | 0.5 | C-0352-N-2 | C01 |
| Fraction_83 | 3.7 | 0.5 | C-0352-N-2 | D01 |
| Fraction_84 | 3.1 | 0.5 | C-0352-N-2 | E01 |
| Fraction_85 | 3.4 | 0.5 | C-0352-N-2 | F01 |
| Fraction_86 | 3.3 | 0.5 | C-0352-N-2 | G01 |
| Fraction_87 | 4.5 | 0.5 | C-0352-N-2 | H01 |
| Fraction_88 | 5.7 | 0.5 | C-0352-N-2 | A02 |
| Fraction_89 | 11.7 | 0.5 | C-0352-N-2 | B02 |
| Fraction_90 | 8.2 | 0.5 | C-0352-N-2 | C02 |
| Fraction_91 | 35.3 | 0.5 | C-0352-N-2 | D02 |
| Fraction_92 | 12.5 | 0.5 | C-0352-N-2 | E02 |
| Fraction_93 | 5.7 | 0.5 | C-0352-N-2 | F02 |
| Fraction_94 | 13.4 | 0.5 | C-0352-N-2 | G02 |
| Fraction_95 | 7.6 | 0.5 | C-0352-N-2 | H02 |
| Fraction_96 | 11.9 | 0.5 | C-0352-N-2 | A03 |
| Fraction_97 | 13.6 | 0.5 | C-0352-N-2 | B03 |
| Fraction_98 | 7.1 | 0.5 | C-0352-N-2 | C03 |
| Fraction_99 | 7.2 | 0.5 | C-0352-N-2 | D03 |
| Fraction_100 | 10.9 | 0.5 | C-0352-N-2 | E03 |
| Fraction_101 | 34.5 | 0.5 | C-0352-N-2 | F03 |
| Fraction_102 | 15.2 | 0.5 | C-0352-N-2 | G03 |
| Fraction_103 | 12.6 | 0.5 | C-0352-N-2 | H03 |
| Fraction_104 | 28.5 | 0.5 | C-0352-N-2 | A04 |
| Fraction_105 | 13.6 | 0.5 | C-0352-N-2 | B04 |
| Fraction_106 | 10.9. | 0.5 | C-0352-N-2 | C04 |
| Fraction_107 | 10.9 | 0.5 | C-0352-N-2 | D04 |
| Fraction_108 | 10.8 | 0.5 | C-0352-N-2 | E04 |
| Fraction_109 | 12.6 | 0.5 | C-0352-N-2 | F04 |
| Fraction_110 | 10.3 | 0.5 | C-0352-N-2 | G04 |
| Fraction_111 | 11.4 | 0.5 | C-0352-N-2 | H04 |
| Fraction_112 | 13.4 | 0.5 | C-0352-N-2 | A05 |
| Fraction_113 | 9.7 | 0.5 | C-0352-N-2 | B05 |
| Postfraction | 151.5 | 0.5 | C-0352-N-2 | C05 |

Aliquots of 0.5 mg of each fraction were pipetted into 96-deep-well plates, which were subjected to receptor translocation assay as described above for activity testing. Remaining amounts of fractions were stored as dry films.

After testing of all fractions the active fractions Fraction_{—}1, Fraction_{—}2, Fraction_{—}3 and Fraction_{—}5, shown in Table S3, were analyzed according to the method listed in Table S4 for purity and the method listed in Table S5 for determination of molecular weight.

TABLE S3

| | Amount_mg after | Purity | Retention time | Compound |
|---|---|---|---|---|
| Comment | Aliquot | >70% | Profiling | M.W. |
| Fraction_l | 10.4 | 96.5 | 20.33 | 364 |
| Fraction_2 | 17.7 | 97.4 | n.n. | 364 |
| Fraction_3 | 23.6 | 98.8 | 20.6 | 366 |
| Fraction_5 | 23.8 | 97.3 | n.n. | 366 |

TABLE S4

| HPLC System | Merck Hitachi | | |
|---|---|---|---|
| Data System | HPLC-Manager D-7000 HSM | | |
| Column | Merck Superspher 60 RP-select B 125 × 4 mm, 4 μm | | |
| Injected Amount | 10 μl | | |
| Flow rate | 1 ml/min | | |
| Detection | ELSD (Sedex 75) | | |
| Mobile Phase: | A: 5 mM ammoniumformate and 0.1% formic acid | | |
| | B: acetonitrile/methanol = 1:1, 5 mM ammoniumformate | | |
| | and 0.1% formic acid (pH 3) | | |
| Gradient | Time [min] | % A | % B |
| | 0.0 | 85 | 15 |
| | 15.0 | 0 | 100 |
| | 18.0 | 0 | 100 |

TABLE S5

| HPLC System | PE series 200 | | |
|---|---|---|---|
| MS System | PE-sciex API 150 | | |
| Data System | Analyst 1.3 | | |
| Stationary phase | Phenomenex Luna C8, 50 × 4.6 mm, 5 μm | | |
| Injected Amount | 30 μl | | |
| Flow rate | 1.5 ml/min | | |
| Detection | (+/(−)-ESI, Fast-Switching-Mode, ELSD (Sedex 75) | | |
| Mobile Phase: | A: 5 mM ammoniumformate and 0.1% formic acid | | |
| | B: acetonitrile/methanol = 1:1, 5 mM ammoniumformate | | |
| | and 0.1% formic acid (pH 3) | | |
| Gradient | Time [min] | % A | % B |
| | 0.0 | 85 | 15 |
| | 5.0 | 0 | 100 |
| | 8.0 | 0 | 100 |

### Structure Analysis of Compounds (A) and (B)

(A)

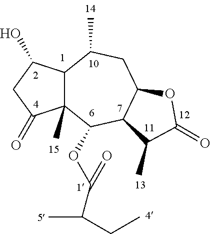

(B)

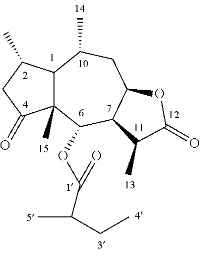

| Compound (A) | | | | Compound (B) | | | |
|---|---|---|---|---|---|---|---|
| | ¹³C-NMR | ¹H-NMR | Position | | ¹³C-NMR | ¹H-NMR | Position |
| 1 | 10.5 q | 1.49 d | 13 | 1 | 10.6 | 1.51 | 13 |
| 2 | 11.3 q | 0.82 t | 4′ | 2 | 15.8 | | 4′ |
| 3 | 15.9 q | 1.14 s | 15 | 3 | 15.9 | 1.16 | 15 |
| 4 | 16.4 q | 1.04 d | 5′ | 4 | 20.2 | | 5′ |
| 5 | 20.1 q | 1.20 d | 14 | 5 | 20.6 | 1.94 | 14 |
| 6 | 23.9 | 2.38 | 9 | 6 | 23.9 | 2.38 | 9 |
| 7 | 26.6 | 1.39 | 3′ | 7 | 39.1 | 2.42 | 10 |
| | | 1.56 | | | | | |
| 8 | 39.2 | 2.43 | 10 | 8 | 40.4 | 3.02 | 11 |
| 9 | 40.4 | 3.01 | 11 | 9 | 46.3 | 2.39 | 3 |
| | | | | | | 2.53 | |
| 10 | 41.1 | 2.26 | 2′ | 10 | 48.2 | 2.81 | 7 |
| 11 | 46.4 t | 2.39 | 3 | 11 | 52.6 | 2.18 | 1 |
| | | 2.53 | | | | | |
| 12 | 48.3 | 2.72 | 7 | 12 | 53.0 | — | 5 |
| 13 | 52.3 | 2.18 | 1 | 13 | 67.9 | 4.59 | 2 |
| 14 | 52.8 s | — | 5 | 14 | 73.3 | 5.38 | 6 |
| 15 | 68.0 d | 4.60 | 2 | 15 | 79.4 | 4.70 | 8 |
| 16 | 73.3 d | 5.30 br s | 6 | 16 | 127.1 s | — | 2′ |
| 17 | 79.3 d | 4.70 | 8 | 17 | 139.4 | 6.03 | 3′ |
| 18 | 175.0 s | — | 1′ | 18 | 166.0 | — | 1′ |
| 19 | 179.0 s | — | 12 | 19 | 179.0 | — | 12 |
| 20 | 216.8 s | — | 4 | 20 | 216.8 | — | 4 |

Compound (A) (arnifoline): ESI-MS (+): m/z 367. The ESI-MS and NMR data indicate the formula of compound (A) is C₂₀H₃₀O₆. The NMR data were compared with the study of Planta Medica 1990, 56, 111-114 [3]; and Planta Medica 2005, 71, 1044-1052 [4], and compound (A) is confirmed to be arnifoline reported in said articles.

Compound (B) (11α-13-dihydroarnifolin B): ESI-MS (+): m/z 365. The ESI-MS and NMR data indicate the formula of compound (A) is C₂₀H₂₈O₆. The NMR data were compared with the study of Planta Medica 1990, 56, 111-114 [3]; and Planta Medica 2005, 71, 1044-1052 [4], and compound (B) is confirmed to be 11α-13-dihydroarnifoline B reported in said articles.

### Tests for BRS3 Activity

### β-Arrestin Mediated Receptor Translocation Assay

The assay was basically performed according that described by Zhang J., et al (1999), THE JOURNAL OF BIOLOGICAL CHEMISTRY 274, 10999-11006.

### Cytosolic Ca⁺⁺ Mobilization Assay

U2OS Cells overexpressing BRS3 were harvested by centrifugation (2 min, 300×g) were resuspended in an assay buffer [24.5 mM HEPES (pH 7.4), 98 mM sodium chloride, 6 mM potassium chloride, 2.5 mM monobasic sodium phosphate, 5 mM sodium pyruvate, 5 mM sodium fumarate, 5 mM sodium glutamate, 2 mM glutamine, 11.5 mM glucose, 1.45 mM calcium chloride, 1.15 mM magnesium chloride, 0.01% soybean trypsin inhibitor, 0.2% (v/v) amino acid mixture, and 0.2% BSA (w/v)] to a concentration of 1.5×10⁶ cells/ml and incubated with 2.5 μM Fura-2/AM (Molecular Probes, Eugene, Oreg.) for 30 min at 37° C. followed by 15 min at 25° C. After two washes with assay buffer, 2 ml of cell suspension were placed in a Delta PTI Scan 1 spectrofluorimeter (Photon Technology International, South Brunswick, N.J.) equipped with a stir bar and water bath (37° C.). Fluorescence was measured at dual excitation wavelengths of 340 nm and 380 nm, using an emission wavelength of 510 nm. Autofluorescence was corrected for by running a sample of unlabeled cells in identical experimental conditions.

### Results of β-Arrestin Mediated Receptor Translocation Assay

Formation of the arrestin-receptor complex occurred as the agonist bind to the receptor and it is able to trace the translocation of this complex from plasma membrane to cytoplasm in a form of endocytoplamic vesicles. Monitoring the distribution and translocation of GFP-labeled arrestin in cell expressing BRS3, we are able to measure the ability of the crude extracts to activate BRS3. Table 2 shows that according to their affinity (EC₅₀) and maximal activation (Eₘₐₓ) on the activation of BRS3, the ethanol extract and ethyl acetate extract of MB1136 have agonist activities (Table 2). These activations are rather specific as none of these two extracts, when used at a concentration up to 10 mg/ml, can induce any receptor translocation or vesicles formation at cells expressing GRPR or NMBR, which are the other members of the bombesin like receptor family sharing 50% homology with BRS3.

TABLE 2

| | BRS3¹ | GRPR/NMBR¹ | EC₅₀² | Eₘₐₓ² |
|---|---|---|---|---|
| MB1136-E³ | OK | No | 0.31 | 40% |
| MB1136-A-01⁴ | OK | No | 0.195 | 70% |

¹0.1 mg/ml of crude extract was used to screen for the ability to activate the receptors.
²Arrestin translocation assays and analysis of dose response relation ship of the crude extracts on the activation of BRS3 were used to determine the affinity and maximal stimulation at BRS3.
³This was ethanol extract.
⁴This was ethyl acetate extract.

The ethanol extract of MB1136 (MB1136-E) were partition with dH₂O, ethyl acetate and hexane. As revealed in Table 3, more than 70% of the activity was extracted in the fraction of ethyl acetate, while little activity was noted in the layer of dH₂O. There is a 4-fold increase in the affinity for activation of BRS3 in the fraction of ethyl acetate layer. The EC₅₀ of the ethyl acetate extract was reduced by a factor of 4, this lead to a 4-fold increase in specific activity (Table 3). The recovery of the activity was more than 75% in this step of purification.

TABLE 3

| | | | Specific | Total | |
|---|---|---|---|---|---|
| | Recovery¹ | EC₅₀ | Activity | Activity³ | Yield⁴ |
| Sample | (mg) | (mg/ml) | (U²/mg) | (U) | (%) |
| MB1136-E | 3860 | 0.126 | 317.5 | 1.2 × 10⁶ | 100 |
| MB1136-E-EA | 640 | 0.03 | 1333 | 8.5 × 10⁵ | 71 |
| MB1136-E-H | 1480 | 0.48 | 83.3 | 1.2 × 10⁵ | 10 |
| MB1136-E-W | 1270 | no | | | |

¹3860 mg of the ethanol extract was subjected to solvent partition with hexane, ethyl acetate and dH²O, the recovery of the materials was indicated
²Unit of activity is defined as the ability to stimulate half-maximal activation of the receptor in a volume of 0.025 ml.
³Total activity is derived by multiplying the specific activity and material recovered.
⁴Yield is derived by the ratio of total activity of the defined fraction and that of MB1136-E, then multiplied by 100%.

Ethyl acetate fraction, MB1136-E-EA, containing the most of the activity, was further subjected to normal phase silica gel column chromatography. The column was washed with hexane, then eluted by steps of increasing ethyl acetate in hexane (5, 10, 20; 30, 40, 50, 75 and 100%), followed by eluting with 20% and 50% of methanol in ethyl acetate. This step of separation resolved the extract into 35 fractions, the amount of compound in each fraction was ranged from 25 g to 200 mg. Judging by the quantity of compound in each fraction, the chromatogram profile revealed 7 peaks, including three fractions that containing more than 10 g/fraction, which are fraction 4, fraction 8 and fraction 16; and 4 minor peaks that contain less than 5 gram/fraction, fraction 20, fraction 22, 25 and 29. Following BRS3 agonist activity assay for each fraction, the chromatogram profile revealed two activity peaks, the major one is fraction 16 and the minor one is fraction 19, which were eluted at 30% and 50% ethyl acetate in hexane, respectively. The agonist activity of the ethyl acetate layer, fraction 15, 16, 17, 18, 19 and 20 were subject to dose dependent analysis to measure the affinity (EC₅₀) and maximal activity (Eₘₐₓ) for BRS3 activation. The results are shown in Table 4. All these fractions were able to activate BRS3 in a dose-dependent and saturable manners, the most pronounced is that there is a 15-fold increase in the affinity of fraction 16 for BRS3 activation, as its EC₅₀ was lower than that of the starting material MB1136-E-EA by a factor of 15. The recovery of total materials from each fraction (109 g) is comparable to the quantity of the starting material (97.5 g), indicating that most of the compound in the starting MB1136-E-EA extract were recovered from the column and collected in each fraction. The recovery of total activity from this step of separation is more than 200% (Table 4), indicating antagonist activity was removed from the extract of MB1136-E-EA.

TABLE 4

| | | | Specific | Total | |
|---|---|---|---|---|---|
| | Recovry¹ | EC₅₀² | Activity³ | Activity⁴ | Yield⁵ |
| Fractions | (g) | (mg/ml) | (U/mg) | (U) | (%) |
| MB1136-E-EA | 97.62 | 0.027 | 1.49 × 10³ | 1.45 × 10⁸ | 100 |
| Fraction 15 | 2.5 | 0.0232 | 1.72 × 10³ | 4.32 × 10⁶ | 2.99 |
| Fraction 16 | 12.59 | 0.0017 | 2.35 × 10⁴ | 2.96 × 10⁸ | 205 |
| Fraction 17 | 3.48 | 0.0059 | 6.78 × 10³ | 2.36 × 10⁷ | 16.3 |
| Fraction 18 | 1.44 | 0.04 | 1.00 × 10³ | 1.44 × 10⁶ | 1 |
| Fraction 19 | 2.39 | 0.01 | 4.05 × 10³ | 9.54 × 10⁶ | 6.6 |
| Fraction 20 | 3.25 | 0.0162 | 2.20 × 10³ | 8.11 × 10⁶ | 5.61 |

¹97.62 g of MB1136-E-EA was subjected silica gel column chromatography and eluted with solvent as described in Experiment
²EC₅₀ was obtained by using nonlinear isothermal analysis of dose dependent receptor activation.
³Unit of activity is defined as the ability to stimulate half-maximal activation of the receptor in a volume of 0.025 ml.
⁴Total activity is derived by multiplying the specific activity and material recovered.
⁵Yield is derived by the ratio of total activity of the defined fraction and that of MB1136-E, then multiplied by 100%.

The major activity peak from this chromatography, fraction 16 and fraction 19, were subjected to reversed phase column chromatography separately as described above. The ability of compounds (1-5) isolated and identified from fraction 16 and the compounds (A) and (B) isolated and identified form fraction 19 to activate BRS3 was analyzed by dose dependent stimulation of β-arrestin mediated receptor translocation assay. These seven compounds are able to activate and stimulate BRS3 translocation in a dose dependent and saturable manner and the EC₅₀ were derived and listed in Table 5.

TABLE 5

| Com- | | | | | | | |
|---|---|---|---|---|---|---|---|
| pound | A | B | 1 | 2 | 3 | 4 | 5 |
| EC₅₀ | 2.7 | 14 | 2.8 | 2.8 | 2.0 | 2.35 | 7.5 |
| Arrestin | μM | μM | μM | μM | μM | μM | μM |
| EC₅₀ | 2.6 | 9.6 | 10 | 12 | 11 | 4.2 | 20 |
| Ca⁺⁺ | μM | μM | μM | μM | μM | μM | μM |

It has been demonstrated that activation of human BRS3 caused cytosolic calcium release (Ryan et al., 1998), we evaluated the effect of compounds (A), (B) and (1-5) on calcium mobilization in the U2OS cells that express human BRS3. As shown in the test results, 25 μg/ml of compound (B) and 6.8 μg/ml of compound (4) stimulated a rapid rise in cytosolic calcium, which reached maximal levels in 13 sec and returning to basal levels in 1 min. This response is strictly dependent on the presence of BRS3, as compounds (B) and (4) were not able to stimulated cytosolic Ca⁺⁺ release in U2OS cells that does not express BRS3. Also as shown in the test results of compounds (A). (B) and (4), both the magnitude of released calcium and the time to reach the peak of the transient were concentration-dependent and saturable. These 11β,13-dihydrohelenalin derivatives caused a detectable response at 0.00034 mg/ml and a maximal 3.6-fold increase at 0.05 mg/ml. Analysis of the dose-response data, by nonlinear iterative curve fitting, the EC₅₀ were derived and listed in Table 5.

To determine the contribution of extracellular calcium to the intracellular calcium mobilization, BRS3 expressed U2OS cells were stimulated with compound (1) to (5) in the Ca⁺⁺ free buffer plus the presence of the Ca⁺⁺-chelating agent EGTA. The results reveal that in the absence of the extracellular Ca⁺⁺, the magnitude of the response was reduce to 20% and the return to basal levels was quicker than that found in the presence of extracellular Ca⁺⁺. To determine the contribution of extracellular calcium to the intracellular calcium mobilization, BRS3 expressed U2OS cells were stimulated with compounds (1-5) in the Ca⁺⁺ free buffer plus the presence of the Ca⁺⁺-chelating agent EGTA. At least 50% of cytosolic Ca⁺⁺ mobilization is derived from intracellular mobilization.

The agonist activity of compounds (A), (B) and (4) are rather selective for BRS3, we perform a dose dependent and receptor activation study for compounds (A), (B), and (4) at the other two members of bombesin receptor family, GRPR and NMBR. As shown in Table 6, the EC₅₀ of compounds (A), (B) and (4) for GRPR and NMBR activation are more than 0.25 mg/ml (Table 6).

TABLE 6

| | EC₅₀ mg/ml¹ | | |
|---|---|---|---|
| Fraction/compound | BRS3 | GRPR | NMBR |
| MB1136-E-EA | 0.027 | >1 | >1 |
| Fraction 19 | 0.03 | >1 | >1 |
| Fraction 16 | 0.0017 | >1 | >1 |
| Compound A | 0.001 | >0.5 | >0.5 |
| Compound B | 0.005 | >0.25 | >0.3 |
| Compound 4 | 0.0013 | >0.3 | >0.3 |

¹EC₅₀ was obtained by using nonlinear isothermal analysis of dose dependent arrestin mediated receptor translocation activation.

### REFERENCES

1. Lee, K. H.; Imakura, Y.; Sims, D.; McPhail, A. T.; Onan, K. D. “Antitumor sesquiterpene lactones from Helenium microcephalum: Isolation of mexicanin-E and structural characterization of microhelenin-B and —C” Phytochemistry 1977, 16, 393-395.
2. Poplawski, J.; Holub, M.; Samek, Z.; Herout, V. “On terpenes. CCIX. Arnicolides—Sesquiterpenic lactones from the leaves of Arnica Montana L.” Coll. Czech. Chem. Commun. 1971, 36, 2189-.
3. Planta Medica 1990, 56, 111-114.
4. Planta Medica 2005, 71, 1044-1052.

1. A method for treating a BRS3 mediated disease in a patient comprising administering to the patient, as an agonist, an amount of a compound having the following structure (1), or a pharmaceutically acceptable salt thereof, effective to modulate a BRS3-mediated biological activity 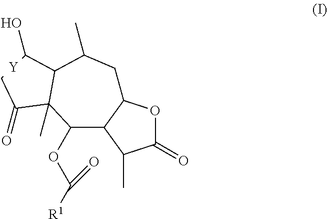
wherein
Y is a single bond or double bound; and
R¹ is —C(CH₃)C₂H₅ or —C(CH₃)═CHCH₃, when Y is a single bound,
R¹ is —C(CH₃)H₂, is —C(CH₃)₂, —C(CH₃)═CHCH₃, —C(CH₃)C₂H₅, or —CH₂C(CH₃)₂,
when Y is a double bound.

2. The method of claim 1 wherein Y is a single bound, and R¹ is —C(CH₃)C₂H₅.

3. The method of claim 1 wherein Y is a single bound, and R¹ is —C(CH₃)═CHCH₃.

4. The method of claim 1 wherein Y is a double bound, and R¹ is —C(CH₃)═CH₂.

5. The method of claim 1 wherein Y is a double bound, and R¹ is —C(CH₃)₂.

6. The method of claim 1 wherein Y is a double bound, and R¹ is —C(CH₃)═CHCH₃.

7. The method of claim 1 wherein Y is a double bound, and R¹ is —C(CH₃)C₂H₅.

8. The method of claim 1 wherein Y is a double bound, and R¹ is —CH₂C(CH₃)₂.

9. The method of claim 1 wherein the disease is metabolic disorders.

10. The method of claim 1 wherein the disease is obesity, glucose intolerance or hypertension.

11. A method for treating a BRS3 mediated disease in a patient comprising administering to the patient, as an agonist, an amount of an extract of Centipeda minima (L.) A. Braun et Aschers. (Compositae) effective to modulate a BRS3-mediated biological activity.

12. The method of claim 11 wherein the extract is prepared by a process comprising the following step:
a) Extracting Centipeda minima (L.) A. Braun et Aschers. (Compositae) with a polar solvent.

13. The method of claim 12 wherein the polar solvent is ethanol, 95% ethanol aqueous solution, or ethyl acetate.

14. The method of claim 13 wherein the preparation process further comprises: b) partitioning the 95% ethanol extract from step a) with ethyl acetate, and recovering ethyl acetate fraction.

15. The method of claim 13 wherein the partitioning in step b) comprises drying the 95% ethanol extract; suspending the dried ethanol extracts in methanol or methanol aqueous solution; partitioning the methanol suspension with hexane, discarding the hexane layer; drying the resultant methanol suspension and re-suspending the dried suspension in water; partitioning the water suspension with ethyl acetate; and concentrating the resultant ethyl acetate layer to obtain the ethyl acetate fraction.

16. The method of claim 14 wherein the preparation process further comprises: c) introducing the ethyl acetate fraction to a normal phase chromatography column; d) eluting with a first eluent of a non-polar solvent such as hexane and with a second eluent in sequence, wherein the second eluent having a polarity of about 30-50 vol % ethyl acetate in hexane; and e) collecting a second eluate from the elution of the column with the second eluent, and removing the second eluent from the second eluate.

17. The method of claim 16 wherein the preparation process further comprises eluting the column with a mixed solvent of ethyl acetate and hexane having 5-20 vol % of ethyl acetate after the elution of the column with the first eluent and before the elution of the column with the second eluent.

18. The method of claim 16 wherein the second eluate from the elution of the column with the second eluent comprises a compound having the structure (I) defined in claim 1.

19. The method of claim 18 wherein the second eluate comprises two compounds defined by Y being a single bound, and R¹ being —C(CH₃)C₂H₅; and by Y being a single bound, and R¹ being —C(CH₃)═CHCH₃.

20. The method of claim 18 wherein the second eluate comprises five compounds defined in claims 4, 5, 6, 7, and 8.

21. The method of claim 18 wherein the second eluate comprises seven compounds defined in claims 2, 3, 4, 5, 6, 7, and 8.

22. The method of claim 11 wherein the disease is metabolic disorders.

23. The method of claim 11 wherein the disease is obesity, glucose intolerance or hypertension.
